(19) United States
(12) Patent Application Publication
(10) Pub. No.: US 2005/0009738 A1
(43) Pub. Date: Jan. 13, 2005

(54) Composition for gene therapy, comprising IL-12 gene

(75) Inventors: Yukio Iwashita, Oita-shi (JP); Seigo Kitano, Beppu-shi (JP); Tadashi Ogawa, Oita-shi (JP); Sigeru Goto, Oita-gun (JP); Katsunori Kawano, Oita-shi (JP)

Correspondence Address: Kendrew H. Colton;Fitch, Even, Tabin & Flannery, Suite 401L, 1801 K Street, N.W., Washington, DC, 20006-1201, US

(73) Assignee: Hisamitsu Pharmaceutical Co., Inc.

(21) Appl. No.: 10/839,142

(22) Filed: May 6, 2004

(30) Foreign Application Priority Data
May 7, 2003 (JP) P2003-129343

(51) Int. Cl. A61K 48/00

(52) U.S. Cl. 514/7; 514/44

(57) ABSTRACT

A composition for gene therapy, comprising IL-12 gene and a nucleic acid carrier, wherein the nucleic acid carrier comprises a polypeptide consisting of diaminobutyric acid and/or a pharmaceutically acceptable salt thereof is prepared, and the composition is delivered to target cells to allow the cells to express IL-12 gene and produce IL-12, which increase a therapeutic effect.

### BACKGROUND OF THE INVENTION

1. Field of the Invention

The present invention relates to the field of gene therapy. More specifically, it relates to a composition for gene therapy comprising the IL-12 gene, and especially to a composition for gene therapy for cancer.

2. Related Background Art

Systems for delivering drugs to target cells or intracellular tissue (Drug Delivery Systems, DDS) are known in pharmacotherapy, but in recent years attention is being focused on gene therapy as a potentially most effective means. Methods for introducing genes (pharmaceutical genes) into cells for gene therapy are now largely classified into two groups.

One group comprises methods using viral vectors, and the other comprises methods using non-viral vectors (particularly artificial nucleic acid carriers such as synthetic polyamino acids). Adenoviruses are commonly employed viral vectors, because of their high infection rate (and therefore high rate of pharmaceutical gene introduction). The latter nucleic acid carriers include synthetic polyamino acids, as well as polymer compounds and liposomes. Methods of preparing and administering such vectors (nucleic acid carriers) are described in detail in the published literature (for example, Muramatsu M., Yamamoto, M., “Shin Idenshi Kogaku [New Gene Engineering] Handbook”, 3rd Revision, Yodosha Publications, Jun. 30, 2000, p.126-134).

A polypeptide based nucleic acid carrier consisting of polydiaminobutyric acid has been developed to overcome the various disadvantages encountered with non-viral vector based nucleic acid carriers (WO00/29031). However, no attempt has been made to apply this specific nucleic acid carrier for actual gene therapy.

Interleukin 12 (IL-12) was initially discovered as a cytokine having an activating action on natural killer (NK) cells, but was later shown to have a proliferating and activating action on killer T cells which exhibit specific cytotoxic activity against tumor cells, and has even been found to potentiate production of interferon γ (IFNγ), which promotes activation of killer T cells. It has therefore been seen as a potentially useful substance for cancer therapy.

However, direct administration of IL-12 to patients causes side-effects including fever and dysorexia, making the treatment unbearable for patients, and therefore IL-12 has not come into use as an anti-tumor agent (Japanese Unexamined Patent Publication HEI No. 10-139670).

### SUMMARY OF THE INVENTION

Despite the confirmed anti-tumor action of IL-12 mentioned above, no effective drug delivery system has been established for it. It is therefore an object of the present invention to provide a composition for gene therapy whereby IL-12 gene is directly introduced into target cells, the gene is efficiently expressed, and IL-12 activity is exhibited in the cells. Specifically, it is an object to provide a composition for gene therapy as cancer therapy, whereby the IL-12 gene is directly introduced into tumor cells to inhibit enlargement or achieve elimination of the tumor.

As a result of much diligent research conducted with the aim of solving the problem described above, the present inventors have completed this invention based on the discovery that by preparing a composition for gene therapy using a carrier consisting of diaminobutyric acid and/or a pharmaceutically acceptable salt thereof as a nucleic acid carrier for delivery of IL-12 gene, a pharmaceutical gene, and by using it, it is possible to efficiently and safely introduce the gene into target cells (particularly tumor cells), and that the introduced gene is expressed for a long period.

In other words, the present invention provides a composition for gene therapy, comprising IL-12 gene and a nucleic acid carrier, wherein the nucleic acid carrier comprises a polypeptide consisting of diaminobutyric acid and/or a pharmaceutically acceptable salt thereof.

The invention further provides a composition for gene therapy, comprising IL-12 gene and a nucleic acid carrier, wherein the nucleic acid carrier comprises a block copolymer of polyethylene glycol and a polypeptide consisting of diaminobutyric acid and/or a pharmaceutically acceptable salt thereof.

The polypeptide in the composition for gene therapy preferably consists of 20 to 280 residues of diaminobutyric acid and/or a pharmaceutically acceptable salt thereof.

The polypeptide in the composition for gene therapy also preferably has 20 to 280 residues and is synthesized by polymerization of a monomer of diaminobutyric acid and/or a pharmaceutically acceptable salt thereof.

The invention still further provides a composition for gene therapy, comprising a complex of an IL-12 gene expression vector and a nucleic acid carrier, wherein the nucleic acid carrier comprises a polypeptide consisting of diaminobutyric acid and/or a pharmaceutically acceptable salt thereof.

The gene therapy of the composition for gene therapy is preferably directed toward treatment of cancer.

The invention still further provides a method of introducing IL-12 gene into target cells (particularly tumor cells), characterized by using the aforementioned composition for gene therapy.

### BRIEF DESCRIPTION OF THE DRAWINGS

FIG. 1 shows the results (by flow cytometry) for a test of gene introduction into B16F10 melanoma cells using a GFP-expressing plasmid/PDBA complex. (a) represents the results for introducing the GFP-expressing plasmid (pDNA) alone as a control, and figure (b) represents the results of introducing the GFP-expressing plasmid/PDBA complex.

FIG. 2 is a graph showing the results of assaying expressed luciferase activity upon injection of different Luc-expressing plasmid/PDBA complexes (pDNA/PDBA) into B16F10 melanoma mouse subcutaneous tumor.

FIG. 3 is a graph showing the results of an anti-tumor test in a B16F10 melanoma subcutaneous tumor mouse model, using an IL-12 gene-expressing plasmid/PDBA complex (PDBA-mIL12).

### DESCRIPTION OF THE PREFERRED EMBODIMENTS

The present invention will now be explained in greater detail by the following examples.

The composition for gene therapy of the invention comprises IL-12 gene and a nucleic acid carrier comprising a polypeptide consisting of diaminobutyric acid and/or a pharmaceutically acceptable salt thereof. Throughout the present specification, the term “IL-12 gene” will sometimes be used synonymously with an expression vector (plasmid) containing the gene. Also, the terms “expression vector” and “expression plasmid” will be used interchangeably.

The nucleic acid carrier used to deliver IL-12 gene as a pharmaceutical gene to target cells is substantially identical to the one described in WO00/29031. Thus, a polypeptide consisting of diaminobutyric acid (polydiaminobutyric acid) as the nucleic acid carrier of the invention has the following structure. 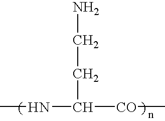
wherein n represents a natural number.

The nucleic acid carrier according to the invention may also be a polypeptide consisting of a pharmaceutically acceptable salt of diaminobutyric acid, in which case it will have the following structure. 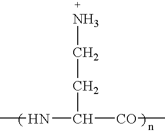
wherein n represents a natural number.

The nucleic acid carrier according to the invention may also contains the two structures shown above in any desired proportion. This encompasses forms of diaminobutyric acid and its pharmaceutically acceptable salts copresent in any desired proportion.

Diaminobutyric acid exists in the D- and L-optical isomers, and the nucleic acid carrier according to the invention encompasses polypeptides of either the D- or L-isomer, as well as any desired combination thereof. Specifically, it includes polypeptides containing poly-L-diaminobutyric acid, poly-D-diaminobutyric acid and poly-DL-diaminobutyric acid.

In the formulas shown above, n represents the number of diaminobutyric acid (or salt) groups, i.e. the number of residues, as the monomers forming the polypeptide. The preferred number of residues in the nucleic acid carrier according to the invention is 10 or greater. It is more preferably 20 or greater, and even more preferably 25 or greater. Also, the number of residues in the nucleic acid carrier according to the invention is preferably 280 or smaller, and even more preferably 250 or smaller. An excessively high number of residues may hinder synthesis or present an inconvenience for handling. An excessively low number of residues will result in inadequate performance as a nucleic acid carrier. The optimal number of residues can be easily selected within the aforementioned range by a person skilled in the art in consideration of the pharmaceutical gene to be used and the properties of components which may be used with it.

As preferred diaminobutyric acid salts there may be mentioned salts of inorganic acids such as hydrochloric acid, sulfuric acid, nitric acid and phosphoric acid or salts of organic acids such as acetic acid, propionic acid, citric acid, lactic acid, oxalic acid, succinic acid, tartaric acid, malonic acid, fumaric acid and malic acid, among which acetic acid salts are particularly preferred, and there is no particular restriction other than the requirement of being a pharmaceutically acceptable salt.

If the polypeptide of the nucleic acid carrier is polydiaminobutyric acid acetate, the number of residues may be represented in terms of molecular weight, based on a molecular weight of 160 for diaminobutyric acid acetate.

As another embodiment of the nucleic acid carrier according to the invention, there may be mentioned a block copolymer structure having polyethylene glycol further bonded to the aforementioned polypeptide consisting of diaminobutyric acid, and the structure is as follows. 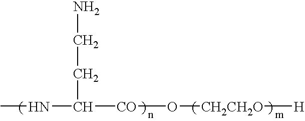
wherein n and m represent natural numbers.

Since the polypeptide has one carboxylate, the above-mentioned block copolymer structure may include the following copolymer. 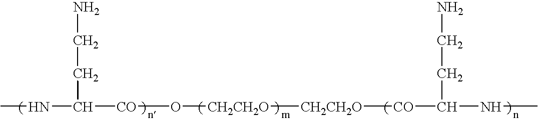
wherein n, n′ and m represent natural numbers.

There are no particular restrictions on the molecular weight for the ethylene glycols (or number of m), but in most cases it is preferably about 200 to 25,000.

The method of synthesizing the nucleic acid carrier of the invention having the structure described above is not particularly restricted, and any of various common publicly known polypeptide synthesis methods may be employed. A particularly preferred synthesis method is described in detail in WO00/29031.

A composition for gene therapy according to the invention may be prepared with the nucleic acid carrier and pharmaceutical gene according to the invention forming a complex in any of various proportions, but a gene/nucleic acid carrier ratio (weight(w)/weight(w)) in the range of 2/1 to 1/50 will produce a particularly notable therapeutic effect, while combination in a gene/nucleic acid carrier (w/w) ratio of 1/1 to 1/30 is more preferred for an even more notable effect. A gene/nucleic acid carrier (w/w) ratio of greater than 1/50 will result in an undesirable increase of the free nucleic acid carrier not associated with the gene, while a ratio of less than 2/1 is not preferred because the gene introduction efficiency will be reduced due to lower affinity with the surfaces of the cells into which the gene is to be introduced. The term “gene” used here refers to DNA, and more precisely it means the expression vector (plasmid) or the like into which the pharmaceutical gene has been introduced.

The source of IL-12 gene, a pharmaceutical gene according to the invention, is not particularly restricted and may be human, cow, rat or mouse, for example.

The pharmaceutical gene may include, in addition to the IL-12 gene, various sequence components such as promoters or enhancers for transcription of the gene, poly A signals, marker genes for mark and/or select gene-introduced cells, viral gene sequences for efficient insertion of the gene into the genomic DNA sequences of the cells, and signal sequences for extracellular secretion and/or intracellular localization of IL-12 as the pharmaceutical product.

The pharmaceutical gene containing IL-12 gene according to the invention is incorporated into a recombinant expression vector and used in a composition for gene therapy as a complex of the recombinant expression vector and the nucleic acid carrier, for introduction into target tissue or cells. The expression vector used for this purpose is not particularly restricted so long as it permits expression of IL-12 gene in the target tissue or cells. As preferred examples there may be mentioned pCAGGS (Gene 108, 193-200 (1991)) pBK-CMV, pcDNA3.1 and pZeoSV (Invitrogen, Stratagene).

The composition for gene therapy of the invention may be prepared by combining the IL-12 gene expression vector and nucleic acid carrier designed for treatment in the manner described above, for example. More specifically, the nucleic acid carrier and the IL-12 gene expression vector may each be dissolved in an appropriate solvent such as water (sterile water), physiological saline, isotonic buffer solution (PBS) or the like, and the solution are mixed and allowed to stand for 10 to 30 minutes to prepare an injection as described below. The ratio of the IL-12 gene expression vector and the nucleic acid carrier is not particularly limited, but as mentioned above, it may be about 0.5-50 μg, and preferably about 1-30 μg of the nucleic acid carrier with respect to 1 μg of the IL-12 gene expression vector.

The method of introducing the composition for gene therapy of the invention into a patient may be a method of gene therapy by autologous transplant (ex vivo gene therapy) which involves first extracting target cells from the body of the patient, introducing the IL-2 gene to the cells, and then returning the cells into the body of the patient. According to the invention, however, gene therapy in which the IL-12 gene is directly administered into the patient for expression of the IL-12 by target cells (particularly tumor cells) (in vivo gene therapy) is preferred.

The method of administering the composition for gene therapy of the invention into the body is not particularly restricted, and may involve any route of administration suitable for the target cells, tissue, organ, etc. For example, the composition may be injected intravenously, intraarterially, subcutaneously, intracutaneously or intramuscularly, or it may be administered directly intralesionally.

The dosage of the composition for gene therapy according to the invention will differ depending on the method and purpose of use, and is very easy for a person skilled in the art to appropriately select and optimize. For example, in the case of administration by injection, the composition is preferably administered at about 0.1 μg/kg to 1000 mg/kg per day, and more preferably about 1 μg/kg to 100 mg/kg per day.

The composition for gene therapy of the invention has an excellent therapeutic effect for patient tissues such as kidney, spleen, lung, bronchi, heart, liver, brain, nerve, muscle, bone marrow, small intestine, colon, large intestine, skin or vascular endothelium. Specific uptake in the liver can be achieved by systemic administration, such as intravenous administration. It is therefore possible to safely and effectively express the pharmaceutical gene specifically in the liver.

The composition for gene therapy of the invention may be pharmacologically evaluated using a common screening panel, and the following animal experiment is particularly suitable when the target disease is cancer. Specifically, an appropriate dose of the composition is administered, at a suitable frequency, to a nude mouse tumor model in which tumor formation has been confirmed. Changes in tumor size are simultaneously observed. The control group used is a group administered the composition without the IL-2 gene. Tumor formation is confirmed in both the group administered the composition for gene therapy of the invention and the control group, and the tumor sizes are measured. Tumor regression in the administered group indicates successful cancer treatment on the experimental animal level.

### EXAMPLES

Synthesis of the nucleic acid carrier used for the example described below was carried out according to the method of K. Vogeler et al. (Helv. Chim. Acta, 43, 270(1960)).

In the production examples, examples and test examples described below, poly-diaminobutyric acid (poly(2,4-diaminobutyric acid)) is abbreviated as PDBA. PDBA also includes all possible isomers thereof. The block copolymer with polyethylene glycol is also abbreviated as PDBApeg (or PDBA-PEG).

### Production Example 1

### Synthesis of Nucleic Acid Carrier

(I) Synthesis of Nγ-carbobenzoxy-DL-diaminobutyric acid NCA (4N-carbobenzoxy-DL-2,4-diaminobutyric acid N-carboxy-anhydride)

(I-1) Synthesis of Nγ-carbobenzoxy-DL-diaminobutyric acid (4N-carbobenzoxy-DL-2,4-diaminobutyric acid):

To 15 g of DL-2,4-diamino-n-butyric acid dihydrochloride (Sigma) dissolved in 75 ml of water was added 11.7 g of basic copper carbonate, and after allowing the mixture to stand it was boiled to reflux and filtered. Then, 16.6 g of sodium hydrogen carbonate and 17.8 ml of carbobenzoxy chloride (Wako Pure Chemical Industries Co., Ltd.) was added to the filtrate and stirred, and the product was obtained as a precipitate. The resultant product was filtered, washed with acetone and diethyl ether, and then dried to give 15 g of Nγ-carbobenzoxy-DL-2,4-diaminobutyric acid copper complex (4N-carbobenzoxy-DL-2,4-diaminobutyric acid copper complex; hereinafter abbreviated as Dba(Z)-Cu).

A portion (11 g) of the obtained complex was placed in a mixture of 9.3 ml of 35% HCl, 22 ml of water and 11 ml of methanol, and the mixture was stirred in the presence of hydrogen sulfide (H₂S) gas. After allowing it to stand at room temperature, the excess hydrogen sulfide was removed and the insoluble portion was removed by filtration. The filtrate was placed under reduced pressure and water and methanol were added for cooling. After adding further methanol, diethylamine was added to adjust the pH of the solution to 7. The precipitated crystals were filtered and separated, diethyl ether was used for washing on a funnel, and drying was performed to give 4 g of product as white crystals. The filtrate was also concentrated and cooled, and the resulting precipitated crystals were filtered and separated, washed on a funnel using diethyl ether, and dried to give 1.5 g of product as white crystals. These crystals were combined to yield a total of 5.5 g of Nγ-carbobenzoxy-DL-diaminobutyric acid (31% yield based on starting amino acid).

(I-2) Synthesis of N-γ-carbobenzoxy-DL-diaminobutyric acid NCA (4N-carbobenzoxy-DL-2,4-diaminobutyric acid N-carboxy-anhydride):

To 5 g of the obtained N-γ-carbobenzoxy-DL-diaminobutyric acid dissolved in 200 ml of tetrahydrofuran (THF) was added 4.5 g of triphosgene (bis(trichloromethyl)carbonate, Aldrich) in 40 ml of THF, and the mixture was stirred at 40° C. for 60 minutes. After removing the solvent under reduced pressure, hexane was added to the resultant crude product for dissolution, and the solution was cooled. The hexane was then thoroughly removed under reduced pressure, and then ethyl acetate was added to the resultant crude product for dissolution and the insoluble portion was removed by filtration. Hexane was added to the filtrate, and the solution was cooled to precipitate the product as white crystals. The precipitated crystals were filtered and separated, and then dried under reduced pressure. The filtrate was also concentrated under reduced pressure and then treated in the same manner to give the product as crystals. The crystals were recrystallized from diethyl ether to give 2.7 g of the purified target product (50% yield).

(II) Polymerization reaction

Nucleic acid carriers with different numbers of residues were obtained by polymerization of the Nγ-carbobenzoxy-DL-diaminobutyric acid NCA obtained above using different proportions of initiator, and then deprotecting the amino-protecting groups.

The number of residues according to the invention was calculated by the following formula (Arieh Yaron et al., Biochim. Biophys. Acta, 69, 397-399, 1963). (The reason for the final multiplication by 0.9 is the approximate 10% reduction in molecular weight under reaction conditions for removal of the protecting groups.) The molecular weight according to the invention is expressed by the formula shown below.

$$Number of residues = Degree of polymerization \\ ( n ) = [ Amount ( moles ) of N γ \text{-} carbobenzoxy \text{-} DL \text{-} diaminobutyric acid NCA ( 10 ) / amount ( moles ) of initiator ] \times yield ( \% ) / 100 \times 0.9$$

$$Molecular weight = n ( degree of polymerization ) \times weight amount of residues ( weight amount of residues = 160 for DBA acetate , as an acetic acid salt )$$

The following synthesis example describes synthesis of the poly-DL-diaminobutyric acid (poly(DL-2,4-diaminobutyric acid) acetate) of Synthesis Example 3 (49 residues) shown in Table 1 and Table 2, but PDBA was also obtained for Synthesis Examples 1 (12 residues), 2 (26 residues), 4 (62 residues), 5 (170 residues), 6 (278 residues) and 7 (348 residues) in the same manner, using the other conditions shown in Table 1. The synthesis conditions for Synthesis Example 8 are also shown in Table 1 and Table 2, for synthesis of polydiaminobutyric acid-PEG obtained by bonding polyethylene glycol (PEG, molecular weight: 1000) at the ends of the polydiaminobutyric acid acetate of Synthesis Example 3.

(II-1) Synthesis of poly-Nγ-carbobenzoxy-DL-diaminobutyric acid (poly(4-N-carbobenzoxy-DL-2,4-diaminobutyric acid):

One gram (3.6 mmol) of Nγ-carbobenzoxy-DL-diaminobutyric acid NCA was dissolved in 19 ml of acetonitrile. After adding 4.38 mg (0.06 mmol) of butylamine as an initiator, the mixture was allowed to stand at 30° C. for 307 hours. The resultant polymer was filtered and washed with acetonitrile. A Soxhlet extractor was used for extraction with diethyl ether, and the extract was dried under reduced pressure to give 0.77 g of poly-Nγ-carbobenzoxy-DL-diaminobutyric acid (91% yield).

(II-2) Synthesis of poly-DL-diaminobutyric acid (poly(DL-2,4-diaminobutyric acid) acetate:

To 0.5 g of poly-N-y-carbobenzoxy-DL-diaminobutyric acid dissolved in 2 ml of trifluoroacetic acid, was added 25% solution of hydrogen bromide in acetic acid, and the mixture was shaken. After standing, diethyl ether was added, the supernatant ether phase was removed with decantation, and then the same procedure was carried out with diisopropyl ether and the supernatant ether phase was removed with decantation. The resultant precipitate was thoroughly dried to solidity under reduced pressure. Sodium acetate and water were added to the solid and the mixture was dialyzed against flowing water using a dialysis tube for ≦1000 molecular weight removal, after which centrifugation was performed at 20,000 G for 1 hour and the precipitate was removed. The solution was lyophilized to give 0.34 g of poly-DL-diaminobutyric acid acetate (91% yield).

TABLE 1
Synthesis conditions for poly(4-N-Carbobenzoxy-DL-2,4-diaminobutyric acid)
by polymerization of 4-N-carbobenzoxy-DL-2,4-diaminobutyric acid NCA

| Synthesis | Initial NCA | Initiator a) | | Solvent c) | Monomer | Temp./ | |
|---|---|---|---|---|---|---|---|
| Example | g(mmol) | mg(mmol) | A/I b) | (ml) | (mol/l) | time | Yield |
| 1 | DL-(Z)diaminobutyric acid | BA | 18 | ACN; 15 | 0.127 | 30° C. | 0.35 g |
| | 0.53 g (1.9) | 7.7(0.105) | | | | 312 hr | (76%) |
| 2 | DL-(Z)diaminobutyric acid | BA | 32 | ACN; 20 | 0.18 | 30° C. | 0.754 g |
| | 1 g (3.6) | 8.21(0.11) | | | | 307 hr | (90%) |
| 3 | DL-(Z)diaminobutyric acid | BA | 60 | ACN; 19 | 0.19 | 30° C. | 0.773 g |
| | 1 g (3.6) | 4.38(0.06) | | | | 307 hr | (91%) |
| 4 | DL-(Z)diaminobutyric acid | BA | 83 | ACN; 20 | 0.127 | 30° C. | 0.491 g |
| | 0.706 g (2.53) | 2.22(0.03) | | | | 312 hr | (83%) |
| 5 | DL-(Z)diaminobutyric acid | BA | 208 | ACN; 20 | 0.127 | 30° C. | 0.539 g |
| | 0.705 g (2.53) | 0.88(0.012) | | | | 312 hr | (91%) |
| 6 | DL-(Z)diaminobutyric acid | BA | 940 | ACN; 19 | 0.19 | 30° C. | 0.764 g |
| | 1.0 g (3.6) | 0.77(0.011) | | | | 360 hr | (91%) |
| 7 | DL-(Z)diaminobutyric acid | BA | 440 | ACN; 19 | 0.19 | 30° C. | 0.739 g |
| | 1.0 g (3.6) | 0.60(0.008) | | | | 360 hr | (88%) |
| 8 | DL-(Z)diaminobutyric acid | PEG 1000 | 46 | ACN; 175 | 0.19 | 30° C. | 0.559 g |
| | 0.78 g (2.8) | 60(0.06) | | | | 307 hr | (76%) |

a) BA: Butylamine; PEG 1000: Polyethylene glycol, MW 1000
b) N-Carboxy anhydride (NCA)/Initiator mol ratio (molar ratio of NCA and initiator)
c) CAN: Acetonitrile

TABLE 2
Synthesis conditions for poly(DL-2,4-diaminobutyric acid) acetate by
deprotection of poly(4-N-carbobenzoxy-DL-2,4-diaminobutyric acid) acetate

| | | | | | | | | | |
|---|---|---|---|---|---|---|---|---|---|
| | | TFA- | | | | | | | |
| Synthesis | Component a) | HbrHAc | | NaOAc(g) | Dialysis | Centrifugation | | No. of | Mol. |
| Example | (g) | (ml) | Temp./time | H₂O(m) | time | temperature | Yield | residues | weight |

| | | | | | | | | | |
|---|---|---|---|---|---|---|---|---|---|
| 1 | Poly(Dba(Z)) | 1.3-1.3 | 24° C. | 0.31 g | 500 cut | 30,000 rpm | 0.115 g | 12 | 1,900 |
| | 0.306 | | 1.8 hr | 20 ml | 17 hr | 4° C., 1 hr | (46%) | | |
| 2 | Poly(Dba(Z)) | 2-2 | 22° C. | 0.5 g | 1000 cut | 30,000 rpm | 0.267 g | 26 | 4,200 |
| | 0.509 | | 2 hr | 25 ml | 17 hr | 4° C., 1 hr | (77%) | | |
| 3 | Poly(Dba(Z)) | 2-2 | 22° C. | 0.5 g | 1000 cut | 30,000 rpm | 0.339 g | 49 | 7,800 |
| | 0.506 | | 2 hr | 25 ml | 17 hr | 4° C., 1 hr | (98%) | | |
| 4 | Poly(Dba(Z)) | 1.5-1.5 | 24° C. | 0.35 g | 1000 cut | 30,000 rpm | 0.139 g | 62 | 9,900 |
| | 0.352 | | 2 hr | 25 ml | 17 hr | 4° C., 1 hr | (58%) | | |
| 5 | Poly(Dba(Z)) | 1.5-1.5 | 24° C. | 0.35 g | 1000 cut | 30,000 rpm | 0.195 g | 170 | 27,200 |
| | 0.379 | | 1.8 hr | 25 ml | 17 hr | 4° C., 1 hr | (75%) | | |
| 6 | Poly(Dba(Z)) | 1.5-1.5 | 24° C. | 0.35 g | 1000 cut | 30,000 rpm | 0.210 g | 278 | 44,500 |
| | 0.379 | | 1.8 hr | 25 ml | 17 hr | 4° C., 1 hr | (81%) | | |
| 7 | Poly(Dba(Z)) | 1.5-1.5 | 24° C. | 0.35 g | 1000 cut | 30,000 rpm | 0.207 g | 348 | 55,700 |
| | 0.379 | | 1.8 hr | 25 ml | 17 hr | 4° C., 1 hr | (80%) | | |
| 8 | Poly(Dba(Z))-PEG | 2-2 | 22° C. | 0.5 g | 1000 cut | 30,000 rpm | 0.254 g | 31 | 6,000 |
| | 0.416 | | 2 hr | 25 ml | 17 hr | 4° C., 1 hr | (78%) | | |

a) Poly(Dba(Z)): Poly(4-N-carbobenzoxy-DL-2,4-diaminobutyric acid)
Poly(Dba(Z))-PEG: Poly(4-N-carbobenzoxy-DL-2,4-diaminobutyric acid)-PEG1000

### Production Example 2

### Preparation of Standard Plasmid/PDBA Complex

In order to evaluate the efficiency of introducing the pharmaceutical gene into target cells, two different standard plasmids were prepared. One was a GFP-expressing (vector) plasmid (eGFP(THDE001, Hisamitsu Pharmaceutical Co., Inc.) and the other was a luciferase-expressing plasmid (Luc(THDF006), Hisamitsu Pharmaceutical Co., Inc.). The PDBA used as the nucleic acid carrier had a molecular weight of 8000. The solution of each plasmid and the PDBA solution were prepared to 2× the desired concentration. The PDBA solution was gradually added dropwise while stirring the plasmid solution about 30 minutes prior to the in vivo or in vitro test, to prepare a plasmid/PDBA complex solution. DMEM medium (Sigma) was used as the solvent. Specifically, 25 μg/ml plasmid solutions were prepared and plasmid/PDBA complex solutions with plasmid concentrations of 12.5 μg/ml were subjected to tests.

### Example 1

### Preparation of Plasmid/PDBA Complex

A murine IL-12 expressing plasmid (pCAGGS-mIL-12) was used as the expression plasmid containing the IL-12 gene, and a pharmaceutical gene plasmid/PDBA complex solution was prepared in the same manner as Production Example 2. The expression plasmid was supplied by Prof. Junichi Miyazaki of Osaka University Graduate School of Medicine.

### Test Example 1

### In vitro Gene Introduction

The standard plasmid/PDBA complex was used to evaluate the efficiency of gene introduction into different mouse tumor cells. Five different mouse tumor cell lines were used for this test (mouse melanoma cell line B16F10, mouse colon tumor cell line Colon26, mouse hepatoma cell line Hepal-6, mouse ascites hepatoma cell line MH134 and mouse ascites hepatoma cell line MH129). The cell lines B16F10, Colon26, Hepal-6, MH134 and MH129 were supplied by Institute of Development, Aging and Cancer, Tohoku University. Cell line Hepal-6 was purchased from the American Type Culture Collection (ATCC).

The test tumor cells (2×10⁶) were collected and rinsed twice with PBS. A cell pellet was prepared from the cells by centrifugation. The standard plasmid solution alone or the standard plasmid/PDBA complex solution was prepared at various concentrations, and added to the cell pellet at 200 μl/10⁶ cells, and after dissolution of the pellet, the solution was allowed to stand for about 1 minute at room temperature. Optimum medium (Gibco Life Technology) was added to 2 ml/10⁶ cells and incubation was carried out for 3-4 hours in a CO₂ incubator (37° C., 5% CO₂)

After centrifugation (1500 rpm, 5 min), each of the cell types was seeded on a 6-well plate (2 ml/well) or a 24-well plate (1 ml/well) and incubated for 48 hours in an incubator.

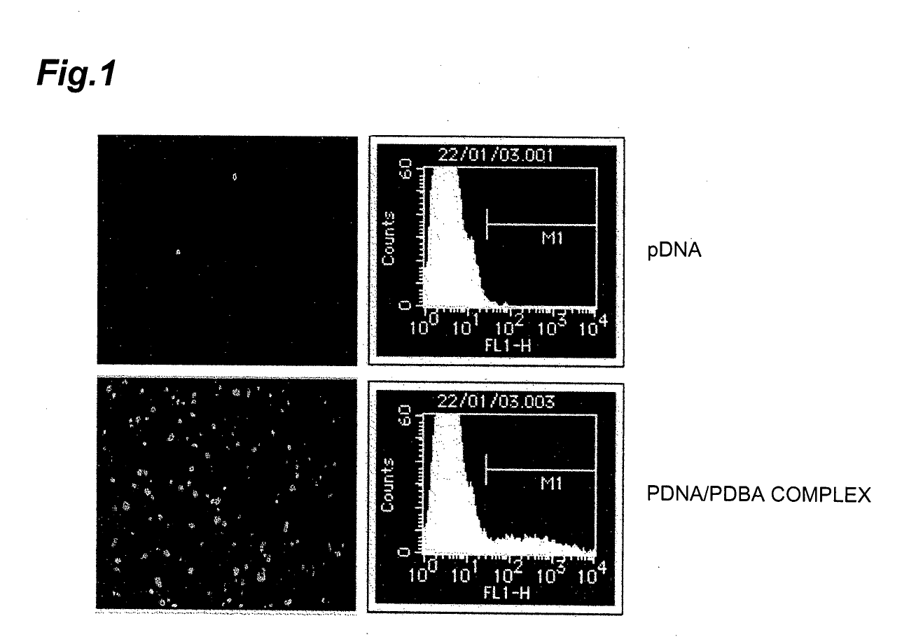

The cells were collected from the plate, the collected cell suspension was fixed with 1% paraformaldehyde, and the eGFP (marker) gene-introduced cells were counted among 10,000 cells using a flow cytometer (FACScan, Becton-Dickinson) and a fluorescent microscope, from which the proportion was calculated. When the introduction efficiency of GFP-expressing plasmid was examined using the mouse melanoma cell line B16F10 as the test cells, for example, the count was approximately 0.3% in the case of the plasmid alone (control), and approximately 11% in the case of the plasmid/PDBA complex. The results of measurement by flow cytometry are shown in FIG. 1. Here, (a) shows the results of introduction using the GFP-expressing plasmid (indicated as pDNA in the figure) as the control, and (b) shows the results of introduction with the GFP-expressing plasmid/PDBA complex (indicated as pDNA/PDBA in the figure). The values calculated for the introduction efficiency for each test cell line are shown in Table 3.

TABLE 3

| Tumor cell line | Cell type | Introduction efficiency |
|---|---|---|
| B16F10 | adherent cells | 10-15% |
| Colon26 | adherent cells | 5-7% |
| Hepal-6 | adherent cells | 7-10% |
| MH134 | suspended cells | 0% |
| MH129 | suspended cells | 0% |

The results in Table 3 show that the introduction efficiency with the plasmid/PDBA complex was 5-15% for the adherent cells (B16F10, Colon26, Hepal-6), while the eGFP gene (plasmid) was not introduced into the suspended cells (MH134 and MH129).

### Test Example 2

### In vivo Gene Introduction

Adherent B16F10 cells were transplanted under the abdominal skin of female C57BL/6 mice (7-8 weeks old) at 2×10⁵/0.1 ml PBS. Gene introduction was attempted after 7 days, when the tumor size reached to approximately 5 mm.

Solutions were prepared with the luciferase (Luc)-expressing plasmid at 100 μg/ml and PDBA at 300 μg/ml, and the PDBA solution was thoroughly agitated and mixed with the plasmid solution. After the mixing, the Luc/PDBA complex solution was injected in the proximity of the mouse subcutaneous tumor at 0.25 ml/tumor within a period of 30 minutes. After 24 hours, the Luc/PDBA complex solution was again injected by the same procedure. That is, the dosage of the Luc/PDBA complex was Luc/PDBA=12.5 μg/37.5 μg, administered twice.

After additional 24 hours (48 hours from the first gene introduction), the protein was extracted from the tumor tissue, and a luciferase assay kit (Toyo Ink Co., Ltd.) and luminometer (Lumit LB9501, Berthold Pty. Ltd.) were used to assay the luciferase activity.

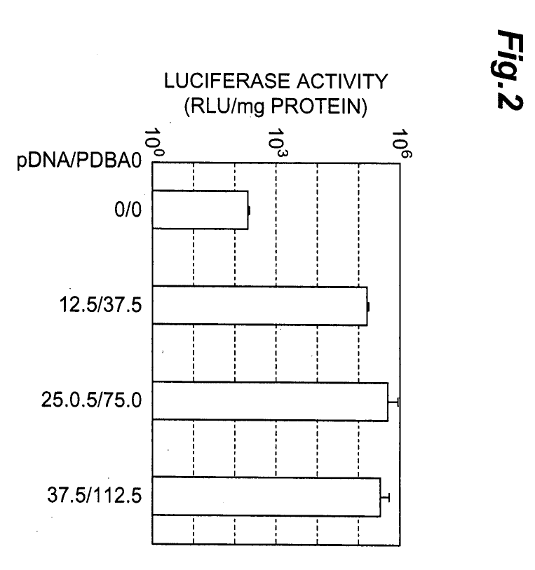

A similar gene introduction test was conducted several times, using different dosages (proportions of Luc and PDBA). The results of assaying the luciferase activity are shown in FIG. 2. The introduction of the gene into the cells occurred stably, independent of dosage, and the optimum conditions were found to be Luc/PDBA=25 μg/75 μg.

### Test Example 3

### IL-12 Gene Introduction into Mice

Based on the results of Test Example 2, a gene therapy test was conducted using a dosage of 25 μg/75 μg of the pCAGGS-mIL-12/PDBA complex in the mouse melanoma subcutaneous tumor model used in Test Example 2 (7 days after tumor seeding). The pCAGGS-mIL-12/PDBA complex (indicated as PDBA-mpIL12 in the drawings below) was administered at the aforementioned dosage to the treatment group (n=6), while PBS, pCAGGS-mIL-12 (indicated as mpIL12 in the drawings below) alone and the Luc/PDBA complex (indicated as PDBA-Luc in the drawings below) were each administered to the control group (n=6). The changes in the sizes of tumors in the mice of each group were observed over time. The maximum diameter (A) and minimum diameter (B) were measured as the tumor sizes, and the tumor volumes were calculated as A×B²/2.

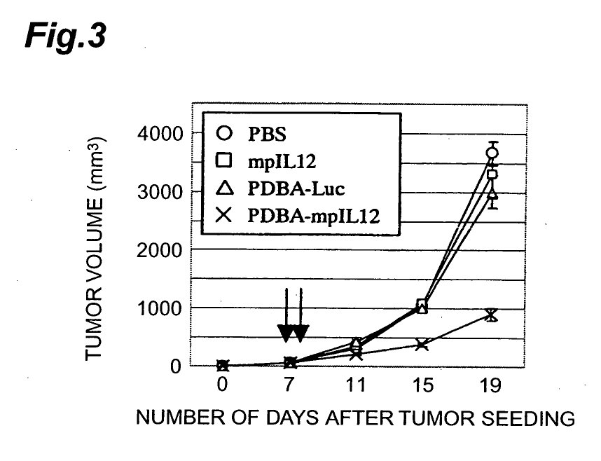

FIG. 3 shows the tumor volumes calculated in the above manner, plotted against the number of days after tumor seeding. The results shown in FIG. 3 demonstrate that tumor growth was significantly (p<0.05) inhibited in the treatment group compared to the control group.

As explained above, the composition for gene therapy of the invention is characterized by comprising IL-12 gene and a polypeptide consisting of an appropriate number of residues of diaminobutyric acid and/or a pharmaceutically acceptable salt thereof, and it allows the gene to be introduced into target cells (particularly tumor cells) in a relatively convenient manner, thereby resulting in expression of the gene in the cells. The IL-2 produced by the gene expression acts on the tumor cells to exhibit an anti-tumor effect.

1. A composition for gene therapy, comprising IL-12 gene and a nucleic acid carrier, wherein the nucleic acid carrier comprises a polypeptide consisting of diaminobutyric acid and/or a pharmaceutically acceptable salt thereof.

2. A composition for gene therapy, comprising IL-12 gene and a nucleic acid carrier, wherein the nucleic acid carrier comprises a block copolymer of polyethylene glycol and a polypeptide consisting of diaminobutyric acid and/or a pharmaceutically acceptable salt thereof.

3. The composition for gene therapy according to claim 1, wherein the polypeptide consists of 20 to 280 residues of diaminobutyric acid and/or a pharmaceutically acceptable salt thereof.

4. The composition for gene therapy according to claim 2, wherein the polypeptide consists of 20 to 280 residues of diaminobutyric acid and/or a pharmaceutically acceptable salt thereof.

5. The composition for gene therapy according to claim 1, wherein the polypeptide has 20 to 280 residues and is synthesized by polymerization of a monomer of diaminobutyric acid and/or a pharmaceutically acceptable salt thereof.

6. The composition for gene therapy according to claim 2, wherein the polypeptide has 20 to 280 residues and is synthesized by polymerization of a monomer of diaminobutyric acid and/or a pharmaceutically acceptable salt thereof.

7. A composition for gene therapy, comprising a complex of an IL-12 gene expression vector and a nucleic acid carrier, wherein the nucleic acid carrier comprises a polypeptide consisting of diaminobutyric acid and/or a pharmaceutically acceptable salt thereof.
